(19) United States
(12) Patent Application Publication
(10) Pub. No.: US 2016/0106327 A1
(43) Pub. Date: Apr. 21, 2016

(54) APPARATUS AND METHOD FOR ACQUIRING BIO-INFORMATION

(71) Applicant: SAMSUNG ELECTRONICS CO., LTD., Suwon-si (KR)

(72) Inventors: Youngzoon Yoon, Hwaseong-si (KR); Hyunsurk Ryu, Hwaseong-si (KR); Kyoobin LEE, Seoul (KR); Jaesoong LEE, Suwon-si (KR); Junhaeng Lee, Hwaseong-si (KR); Seunghoon HAN, Seoul (KR)

(73) Assignee: SAMSUNG ELECTRONICS CO., LTD., Suwon-si (KR)

(21) Appl. No.: 14/884,019

(22) Filed: Oct. 15, 2015

(30) Foreign Application Priority Data
Oct. 15, 2014 (KR) 10-2014-0139065

(51) Int. Cl. A61B 5/021; A61B 5/026; A61B 5/00

(52) U.S. Cl. CPC A61B 5/02108; A61B 5/681; A61B 5/0261; A61B 5/742

(57) ABSTRACT

An apparatus for acquiring bio-information includes a light source configured to radiate a laser beam to a region of interest including a blood vessel; a sensor configured to sense, from the region of interest, a change of a laser speckle generated by the radiated laser beam; and a controller configured to obtain a bio-signal indicating a change in a blood flow in the blood vessel based on the sensed change of the laser speckle.

### CROSS-REFERENCE TO RELATED APPLICATIONS

This application claims priority from Korean Patent Application No. 10-2014-0139065, filed on Oct. 15, 2014 in the Korean Intellectual Property Office, the disclosure of which is incorporated herein in its entirety by reference.

### BACKGROUND

1. Field

Apparatuses and methods consistent with exemplary embodiments relate to acquiring bio-information based on a motion of a living body.

2. Description of the Related Art

Information about a blood pressure or a blood flow is used as bio-information for identifying a personal health state. In general, a blood pressure indicates a cardiac output, elasticity of a blood vessel, a physiological change of an object.

The blood pressure is measured in an invasive method and a non-invasive method. A typical invasive method is to directly measure a pressure of a blood vessel by inserting a catheter into the blood vessel. However, this method has a risk of arterial bleeding. Also, since application of invasion is necessary, the method may not be frequently or conveniently used to examine a health state.

Also, the non-invasive method includes auscultation, oscillometry, tonometry, etc. The auscultation and oscillometry are methods for measuring a blood pressure by using a cuff while applying a pressure to a human body. According to the auscultation method, a systolic pressure and a diastolic pressure of the heart of a target patient may be measured by measuring Korotkoff sound that is generated as a cuff gradually constricts. Also, the oscillometry method is to measure an actual pressure change occurring in a cuff as the cuff constricts. The tonometry method is to measure a change in the intraarterial pressure by using a sensor placed on the artery in a state in which an influence of a blood vessel wall tension is removed by pressing the artery enough to make a flat portion in an external carotid artery having a bone supporter, such as a radial artery. However, the above-described non-invasive methods are not appropriate for measuring in real time a change in the blood pressure of an individual.

### SUMMARY

Exemplary embodiments address at least the above problems and/or disadvantages and other disadvantages not described above. Also, the exemplary embodiments are not required to overcome the disadvantages described above, and may not overcome any of the problems described above.

One or more exemplary embodiments provide an apparatus and method for acquiring bio-information based on a motion of a living body.

According to an aspect of an exemplary embodiment, there is provided an apparatus for acquiring bio-information including: a light source configured to radiate a laser beam to a region of interest including a blood vessel, a sensor configured to sense a change of a laser speckle generated by the radiated laser beam, from the region of interest, and a controller configured to obtain a bio-signal indicating a change in a blood flow in the blood vessel based on the sensed change of the laser speckle.

The sensor may sense whether a strength of an optical signal corresponding to the laser speckle increases or decreases.

The sensor may include a dynamic vision sensor (DVS).

The sensor may enter a standby mode when the change of the laser speckle is not sensed for a predetermined time.

The controller may estimate a blood pressure by using the obtained bio-signal.

The controller may generate a waveform indicating a change in the volume of the blood vessel based on the change in the blood flow and the sensed change of the laser speckle, and determine a blood pressure of the blood vessel based on the at least one of parameters of the waveform.

The controller may determine the blood pressure by applying the at least one of parameters to a blood pressure estimation model, and the blood pressure estimation model may be generated based on a correlation between the at least one of parameters and a reference blood pressure.

The controller may generate a waveform indicating a change in a volume of the blood vessel based on the sensed change of the laser speckle, generate a photoplethysmogram (PPG) waveform indicating the volume of the blood vessel based on the waveform, and determine the blood pressure based on the PPG waveform.

The controller may determine an acceleration of the blood flow based on a correlation between the sensed change of the laser speckle and a reference blood flow acceleration.

The apparatus may further include a display configured to display an image indicating the determined acceleration of the blood flow.

According to an aspect of another exemplary embodiment, there is provided a method of acquiring bio-information including: radiating a laser beam to a region of interest (ROI) including a blood vessel, sensing a change of a laser speckle generated by the radiated laser beam, from the region of interest, and obtaining a bio-signal indicating a change in a blood flow in the blood vessel based on the sensed change of the laser speckle.

The sensing the change of the laser speckle change include sensing whether a strength of the laser speckle is increased or decreased may be sensed.

The method may further include entering a standby mode in response to the change of the laser speckle not being detected for a predetermined period of time.

The method may further include a blood pressure of the blood vessel based on the obtained bio-signal.

The method may further include: generating a waveform indicating a change in a volume of the blood vessel based on the change in the blood flow and the sensed change of the laser speckle; and determining a blood pressure of the blood vessel based on at least one of parameters of the waveform.

The determining the blood pressure includes s applying the at least one of parameters to a blood pressure estimation model, wherein the blood pressure estimation model is generated based on a correlation between the at least one of parameters and the blood pressure.

The method may further include: generating a waveform indicating a change in a volume of the blood vessel based on the sensed change of the laser speckle; generating a photoplethysmogram (PPG) waveform indicating the volume of the blood vessel based on the waveform; and determining a blood pressure based on the PPG waveform.

The method may further include determining an acceleration of the blood flow based on a correlation between the sensed change of the laser speckle and a reference blood flow acceleration.

The method may further include displaying an image indicating the estimated acceleration of the blood flow.

According to an aspect of another exemplary embodiment, there is provided a non-transitory computer readable storage medium storing a program that is executable by a computer to perform the method of acquiring the bio-information.

According to another aspect of an exemplary embodiment, there is provided an apparatus for obtaining bio-information including: an optical sensor configured to capture an image of a skin surface of a subject within a region of interest (ROI) and generate data on a portion of the image that has a relative change of a light intensity in relation to a remaining portion of the image; and a controller configured to obtain the bio-information indicating a change in a blood vessel within the ROI based on the generated data.

The optical sensor includes a dynamic vision sensor (DVS) and the generated data reflects the change of the light intensity which is caused by a blood flow of the blood vessel within the ROI.

### BRIEF DESCRIPTION OF THE DRAWINGS

The above and/or other aspects will become apparent and more readily appreciated from the following description of exemplary embodiments, taken in conjunction with the accompanying drawings, in which:

FIG. 1 is a view for explaining an apparatus for acquiring bio-information by using a laser speckle according to an exemplary embodiment;

FIG. 2 illustrates an example of an apparatus for acquiring bio-information according to an exemplary embodiment;

FIG. 3 is a block diagram illustrating a structure of an apparatus for acquiring bio-information according to an exemplary embodiment;

FIG. 4 is a flowchart for explaining a method of acquiring bio-information according to an exemplary embodiment;

FIG. 5A is an image showing a change in the laser speckle sensed from a region of interest (ROI) according to an exemplary embodiment;

FIG. 5B is a graph showing the strength of a laser speckle reflected from a particular area according to time according to an exemplary embodiment;

FIG. 6 is a flowchart for explaining a method of estimating a blood pressure according to an exemplary embodiment;

FIGS. 7A, 7B, and 7C respectively illustrate a speckle distribution pattern (first pattern) when a blood vessel constricts, a speckle distribution pattern (second pattern) when the blood vessel dilates, and a comparison between the first pattern and the second pattern, according to an exemplary embodiment;

FIGS. 8A and 8B illustrate changes in the laser speckle according to an exemplary embodiment;

FIGS. 9A and 9B are graphs respectively showing a waveform indicating a change in the volume of a blood vessel obtained in the apparatus for acquiring bio-information according to an exemplary embodiment and a photoplethysmogram (PPG) waveform, that is, a waveform indicating the volume of a blood vessel;

FIGS. 10A, 10B, and 10C are graphs respectively showing first to third waveforms according to an exemplary embodiment;

FIG. 11 is a flowchart for explaining a method of acquiring information about a blood flow according to an exemplary embodiment; and

FIGS. 12A, 12B, and 12C are views for explaining a method of acquiring information about a blood flow according to an exemplary embodiment.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

Expressions such as “at least one of,” when preceding a list of elements, modify the entire list of elements and do not modify the individual elements of the list.

When a part may “include” a certain constituent element, unless specified otherwise, it may not be construed to exclude another constituent element but may be construed to further include other constituent elements. Terms such as “˜portion”, “˜unit”, “˜module”, and “˜block” stated in the specification may signify a unit to process at least one function or operation and the unit may be embodied by hardware, software, or a combination of hardware and software.

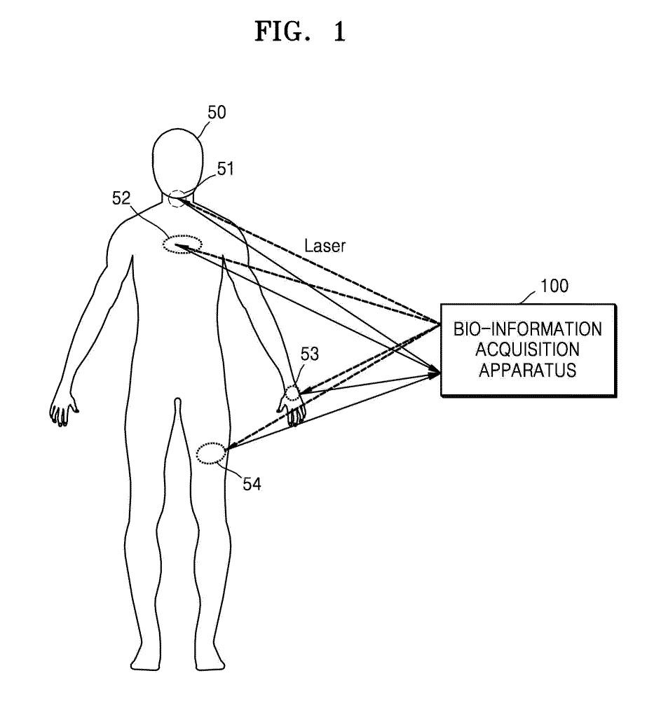

FIG. 1 is a view for explaining an apparatus 100 for acquiring bio-information by using a laser speckle according to an exemplary embodiment.

Referring to FIG. 1, the bio-information acquisition apparatus 100 irradiates a region of interest (ROI) of an object 50 using a laser beam. The object 50 may be a human or an animal, and the ROI may include a part having movement of a body of a human or an animal, for example, a neck part 51, a chest part 52, a wrist part 53, a leg part 54, etc. However, the present exemplary embodiment is not limited thereto.

When a laser beam is radiated to the ROI, the bio-information acquisition apparatus 100 may sense a change of a laser speckle from the ROI. The laser speckle is an intensity pattern like an irregular speckle or an irregular pattern, generated by an interference phenomenon or a scattering phenomenon when a laser beam having interference properties is radiated onto a body. The laser speckle may appear to be a form of scattered spots.

The bio-information acquisition apparatus 100 may sense a change in the intensity of an optical signal corresponding to the laser speckle. For example, the bio-information acquisition apparatus 100 may include a dynamic vision sensor (DVS), and a change of the speckle may be sensed by using the DVS. The DVS is a sensor for sensing a change in the intensity (amount) of light, that is, a change in the intensity of an optical signal. When there is no change in the intensity of light, the DVS may enter a standby mode.

For example, while an image sensor of a complementary metal-oxide semiconductor (CMOS) or (charge-coupled device) CCD type senses analog information of the intensity of light that constitutes an image, the DVS may detect a change in the intensity of light, instead of the intensity of light, as digital information. For example, when the intensity of light increases, it may be presented to be +1, when there is no change in the intensity of light, it may be presented to be 0, and when the intensity of light decreases, it may be presented to be −1.

Accordingly, when there is a motion in the ROI onto which a laser beam is radiated and thus a distribution of a laser speckle changes, the intensity of an optical signal corresponding to the laser speckle change and thus the DVS may sense a change in the intensity of an optical signal. The bio-information acquisition apparatus 100 may obtain information about a motion of the ROI by using the change in the intensity of an optical signal corresponding to the sensed laser speckle (hereinafter, referred to as the laser speckle change).

For example, when a person produces voice, the vocal cords are vibrated. In this state, the bio-information acquisition apparatus 100 may radiate a laser beam onto the neck part 51 where the vocal cords are located. The DVS may sense the laser speckle change due to the motion of the vocal cords. The bio-information acquisition apparatus 100 may obtain the information about a motion of the vocal cords based on the sensed laser speckle change.

Also, when a person breathes, lungs are repeatedly constricted and dilated. In this state, the bio-information acquisition apparatus 100 may radiate a laser beam onto the chest part 52 where the lungs are located and then the DVS may sense the laser speckle change due to the motions of lungs. The bio-information acquisition apparatus 100 may obtain information about the motions of lungs and information about respiration of the object, based on the sensed laser speckle change.

Also, blood flows in blood vessels by contraction and relaxation of the heart. In this state, the bio-information acquisition apparatus 100 may radiate a laser beam onto the wrist part 53 including blood vessels through which blood flows, and the DVS may sense the laser speckle change due to the blood flow. The bio-information acquisition apparatus 100 may obtain information about the blood flow based on the sensed laser speckle change.

Also, the bio-information acquisition apparatus 100 may irradiate not only the wrist part 53, but also the leg part 54, using a laser beam. The DVS may sense the laser speckle change due to the blood flow of the leg part 54. The bio-information acquisition apparatus 100 may obtain information about a blood flow in the leg portion 54 based on the sensed laser speckle change. In addition, the bio-information acquisition apparatus 100 may radiate a laser beam onto all body parts such as an eye, a forehead, a palm, etc., where a motion exists, and may sense the laser speckle change.

The bio-information acquisition apparatus 100 according to the present exemplary embodiment may acquire bio-information of an object by using the laser speckle change sensed from the ROI. In the following description, for convenience of explanation, it is assumed that the ROI is a wrist part including a blood vessel.

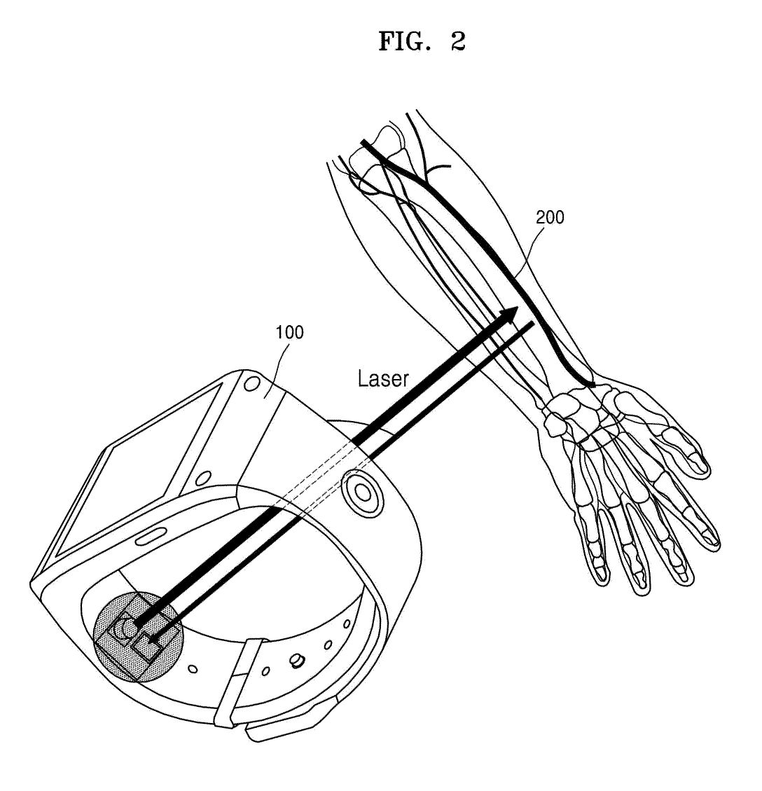

FIG. 2 illustrates an example of the bio-information acquisition apparatus 100 according to an exemplary embodiment.

Referring to FIG. 2, the bio-information acquisition apparatus 100 may be a wrist watch type or wrist band type wearable device. However, the present exemplary embodiment is not limited thereto, and the bio-information acquisition apparatus 100 may be wearable devices of various types such as a glasses type, a ring type, or a necklace type.

As illustrated in FIG. 2, the bio-information acquisition apparatus 100 that may be worn around a wrist part may acquire bio-information of the object by radiating a laser beam onto the wrist part in a state of being worn around the wrist part of a user and sensing a laser speckle change generated by the radiated laser beam in the wrist part.

The bio-information of an object may include information about a blood pressure of the object or information about a blood flow of the object. The blood pressure signifies a pressure affecting a blood vessel wall when blood output from the heart flows through the blood vessel. The blood pressure may be divided into an arterial blood pressure, a capillary blood pressure, a venous blood pressure, etc. according to the name of a blood vessel. The arterial blood pressure varies according to the heart beats. Also, the blood pressure includes both of a systolic blood pressure when the ventricle of the heart constricts and thus blood is pushed out into the arteries, and a diastolic blood pressure when the ventricle of the heart dilates and thus blood is not pushed out.

Referring to FIG. 2, the bio-information acquisition apparatus 100 may measure an arterial blood pressure by radiating a laser beam onto a surface of skin close to a radial artery 200 in a contact or non-contact manner. As illustrate in FIG. 2, if a blood pressure is measured on the surface of skin of the wrist part where the radial artery 200 passes, the measurement may be least affected by external factors, for example, a thickness of skin tissue in the wrist part, which cause errors in the measurement of a blood pressure. Also, the radial artery 200 is known to be a blood vessel where a blood pressure may be accurately measured compared to other blood vessels in the wrist part.

However, the present exemplary embodiment is not limited thereto, and the bio-information acquisition apparatus 100 may measure a blood pressure by using a blood vessel other than the radial artery 200 in the other parts of the wrist part.

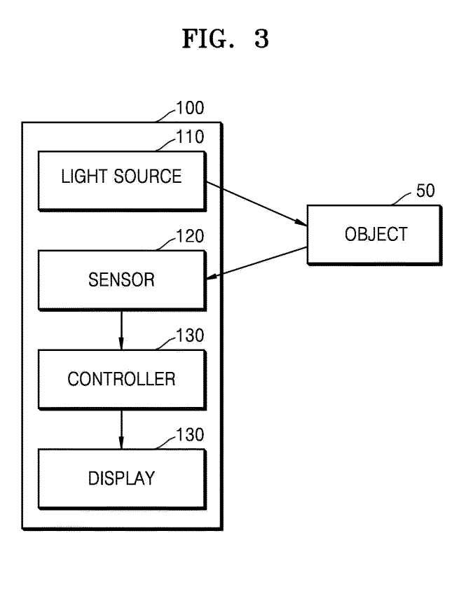

FIG. 3 is a block diagram illustrating a structure of the bio-information acquisition apparatus 100 according to an exemplary embodiment.

Referring to FIG. 3, the bio-information acquisition apparatus 100 may include a light source 110, a sensor 120, and a controller 130. However, the bio-information acquisition apparatus 100 may include more elements other than the elements illustrated in FIG. 3.

The light source 110 may radiate a laser beam toward a blood vessel, for example, a radial artery, in the ROI, for example, the wrist part, of the object. The light source 110 may include at least one laser diode device that radiates a laser beam. The light source 110 may include a laser diode driver that controls laser oscillation in addition to the laser diode device.

The sensor 120 senses a change of a laser speckle generated by a scattering phenomenon or an interference phenomenon of the radiated laser beam, from the ROI, for example, the wrist part. The laser speckle denotes an irregular light intensity pattern generated by the scattering phenomenon or the interference phenomenon when a laser beam having interference onto a body. The laser speckle may be indicated in the form of scattered dots on a photographed image with respect to the ROI onto which the laser beam is radiated.

The sensor 120 may include the DVS, and may sense a change in the intensity of an optical signal corresponding to the laser speckle. The DVS is a sensor that senses a change in the intensity of light or an optical signal. When there is no change in the intensity of an optical signal, the DVS may enter a standby mode.

As illustrated in FIG. 1, while the image sensor of a CMOS or CCD type that detect analog information of the intensity or amount of light that constitutes an image, the DVS may detect a change in the intensity of light, instead of the intensity of light, as digital information. For example, when the intensity of light increases, it may be presented to be +1, when there is no change in the intensity of light, it may be presented to be 0, and when the intensity of light decreases, it may be presented to be −1. Accordingly, the sensor 120 may sense not the intensity of an optical signal corresponding to the laser speckle, but a change of the intensity of the optical signal, that is, the laser speckle change.

When a motion exists in the ROI onto which a laser beam is radiated, the distribution pattern of a laser speckle changes and thus the intensity of an optical signal corresponding to the laser speckle changes. Accordingly, when a motion exists in the ROI, the sensor 120 may sense the laser speckle change. For example, a blood flow rate in a blood vessel changes according to the contraction and relaxation of the heart and thus the blood vessel constricts or dilates according to a change in the blood flow rate. Accordingly, when the bio-information acquisition apparatus 100 radiates a laser beam onto a portion where a blood vessel is located, the distribution pattern of the laser speckle may change according to the constriction and dilation of the blood vessel. The sensor 120 may sense the change in the distribution pattern of the laser speckle.

In contrast, when there is no motion in the ROI, the sensor 120 enters a standby mode and thus a power consumption amount of the bio-information acquisition apparatus 100 may be reduced.

The controller 130 may obtain a bio-signal that indicates a change in the volume according to the constriction and dilation of a blood vessel, for example, a radial artery, by using the sensed change in the distribution pattern of the laser speckle, and may estimate the blood pressure based on the obtained bio-signal.

The controller 130 may obtain a bio-signal based on a change in the intensity of an optical signal corresponding to the laser speckle sensed by the sensor 120. For example, the controller 130 may obtain a bio-signal by analyzing the laser speckle change that is changed according to a change in the volume of a blood vessel. Since the change in the volume of a blood vessel corresponds to the change in the blood flow rate of the blood vessel, the bio-signal obtained by analyzing the laser speckle change may be a signal indicating the change in the blood flow rate. In other words, the obtained bio-signal may be a signal in a differential form of a photoplethysmogram (PPG) signal.

The controller 130 may estimate a systolic blood pressure and a diastolic blood pressure by using a predetermined algorithm for calculating a blood pressure from the signal indicating a change in a blood flow rate, that is, the signal in a differential form of a PPG signal. For example, the controller 130 may extract at least one parameter from the obtained bio-signal, and may estimate a systolic blood pressure and a diastolic blood pressure, based on a correlation between the extracted parameters and the blood pressure.

Also, the controller 130 may generate a speckle change image based on the laser speckle change sensed by the sensor 120. The controller 130 may analyze a change in a spatio-temporal distribution pattern of the laser speckle, based on a change in the intensity of an optical signal corresponding to the laser speckle sensed by the sensor 120. Also, the controller 130 may analyze a spatio-temporal correlation with respect to the ROI by using the change in the distribution pattern of the laser speckle. Further, the controller 120 may estimate an acceleration of a blood flow, which is the rate of change of velocity of the blood flow. For example, the controller 130 may estimate an acceleration of a blood flow by using a predetermined algorithm for analyzing the spatio-temporal correlation and the acceleration of a blood flow, and may generate an image that shows an accelerative change of the distribution of an estimated blood flow. Also, the controller 130 may obtain a velocity distribution of a blood flow based on the accelerative change of a blood flow.

The bio-information acquisition apparatus 100 may further include a display 140. The display 140 may display bio-information of a user. For example, the display 140 may display information about a blood pressure, numerical information about a minimum blood pressure and a maximum blood pressure of a user, numerical information about a systolic blood pressure and a diastolic blood pressure of a user, or information about whether a current blood pressure state is normal or abnormal. Also, the display 140 may display an image indicating a distribution of an accelerative change of a blood flow or an image indicating a distribution of velocity of a blood flow.

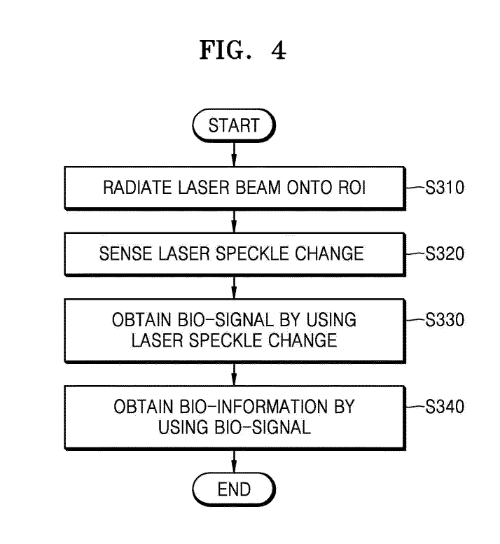

FIG. 4 is a flowchart for explaining a method of acquiring bio-information according to an exemplary embodiment.

Referring to FIG. 4, the bio-information acquisition apparatus 100 may radiate a laser beam to the ROI (operation S310).

The ROI may include any movable part of a body of a human or an animal, for example, a neck part where the vocal cords move, a chest part where lungs move due to inspiration, a wrist part where a blood vessel moves, etc. However, the present inventive concept is not limited thereto. For convenience of explanation, the ROI is described below as a wrist part including a blood vessel.

When the bio-information acquisition apparatus 100 radiates a laser beam onto a wrist part, a laser speckle may be generated due to an interference phenomenon or a scattering phenomenon. Also, the laser speckle changes due to a motion of the blood vessel included in the wrist part, for example, constriction and dilation of a blood vessel are generated due to a change in a blood flow rate. The laser speckle change may signify a change of a distribution pattern of the laser speckle. Accordingly, the laser speckle change may signify a change in the intensity of an optical signal corresponding to the laser speckle.

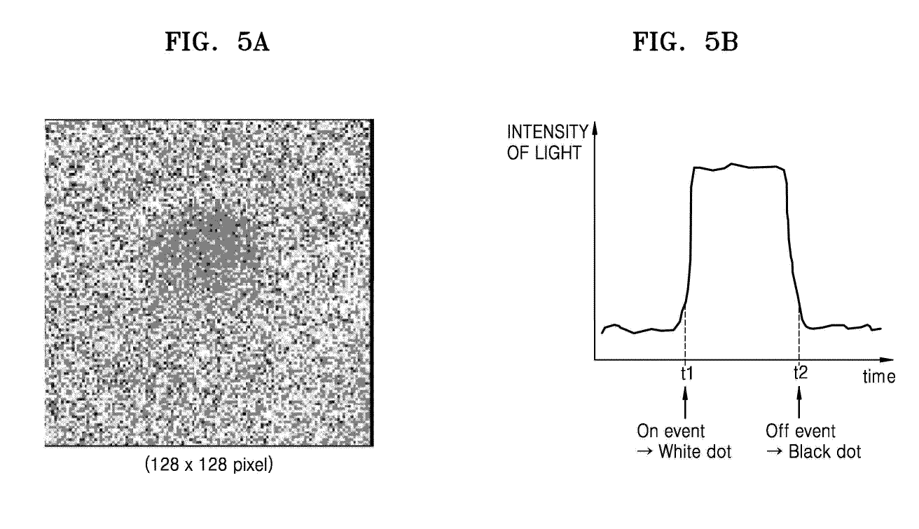

The bio-information acquisition apparatus 100 may sense the laser speckle change from the ROI (operation S320), which is described in detail with reference to FIGS. 5A and 5B.

FIG. 5A is an image showing a change in the laser speckle sensed from the ROI according to an exemplary embodiment. As illustrated in FIG. 5A, the bio-information acquisition apparatus 100 may generate information of a laser speckle change sensed for each pixel as an image including any one of a black dot, a white dot, and a gray dot. For example, when the strength of a laser speckle sensed from any one pixel increases, the bio-information acquisition apparatus 100 may present the pixel as a white dot. Also, when the strength of a laser speckle sensed from any one pixel decreases, the bio-information acquisition apparatus 100 may present the pixel as a black dot. Also, when there is no change in the strength of a laser speckle sensed from any one pixel, the bio-information acquisition apparatus 100 may present the pixel as a gray dot. However, the present exemplary embodiment is not limited thereto.

FIG. 5B is a graph showing the strength of a laser speckle reflected from a particular area according to time, according to an exemplary embodiment. FIG. 5B shows that a change in the intensity of the sensed laser speckle is less than a critical value when a time variable is between 0 and t1. Accordingly, the bio-information acquisition apparatus 100 may present a pixel corresponding to a particular area as a gray dot. Also, when a change in the strength of a laser speckle is less than the critical value, the bio-information acquisition apparatus 100 may enter a standby mode.

Also, the strength of a laser speckle increases at t1 and an amount of the increase is equal to or greater than the critical value (On event). Accordingly, the bio-information acquisition apparatus 100 may present a pixel corresponding to a particular area as a white dot. Also, a change in the strength of a laser speckle is less than the critical value in a section from t1 to t2. Accordingly, the bio-information acquisition apparatus 100 may present a pixel corresponding to a particular area as a gray dot.

Also, the strength of a laser speckle decreases at t2 and an amount of the decrease is equal to or greater than the critical value (Off event). Accordingly, the bio-information acquisition apparatus 100 may present a pixel corresponding to a particular area as a black dot.

The bio-information acquisition apparatus 100 may obtain a bio-signal by using the laser speckle change (operation S330), and may obtain bio-information by using the bio-signal (operation S340).

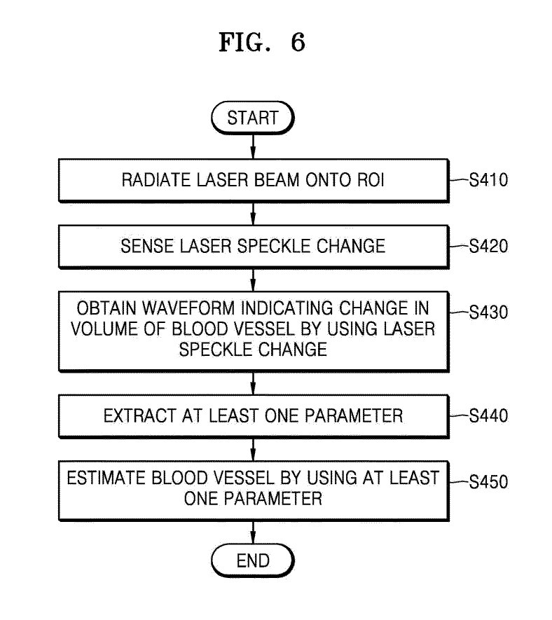

For example, the bio-information acquisition apparatus 100 may obtain a bio-signal indicating a change in the volume according to constriction and dilation of a blood vessel, for example, a radial artery, by using the sensed laser speckle change, and may estimate a blood pressure based on the obtained bio-signal, which is described below in detail with reference to FIG. 6.

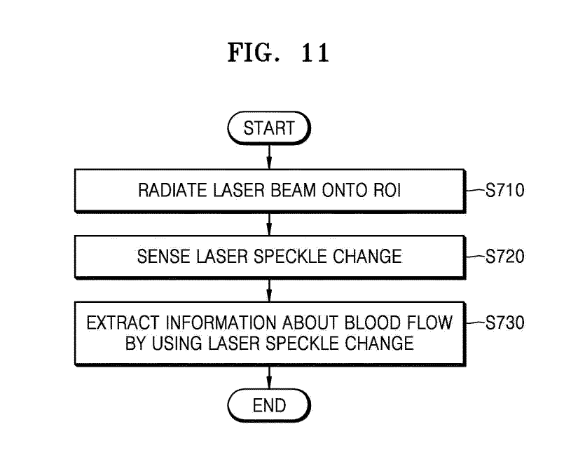

Also, the bio-information acquisition apparatus 100 may obtain spatio-temporal correlation with respect to the ROI by using the change in the distribution pattern of the sensed laser speckle, and may estimate an accelerative change of a blood flow based on the obtained spatio-temporal correlation, which is described below in detail with reference to FIG. 11.

FIG. 6 is a flowchart for explaining a method of estimating a blood pressure according to an exemplary embodiment.

Since operations S410 and operation S420 of FIG. 6 are substantially the same as operations S310 and operation S320 of FIG. 4, detailed descriptions thereof are omitted.

The bio-information acquisition apparatus 100 may obtain a waveform indicating a change in the volume of a blood vessel by using the sensed laser speckle change (operation S430).

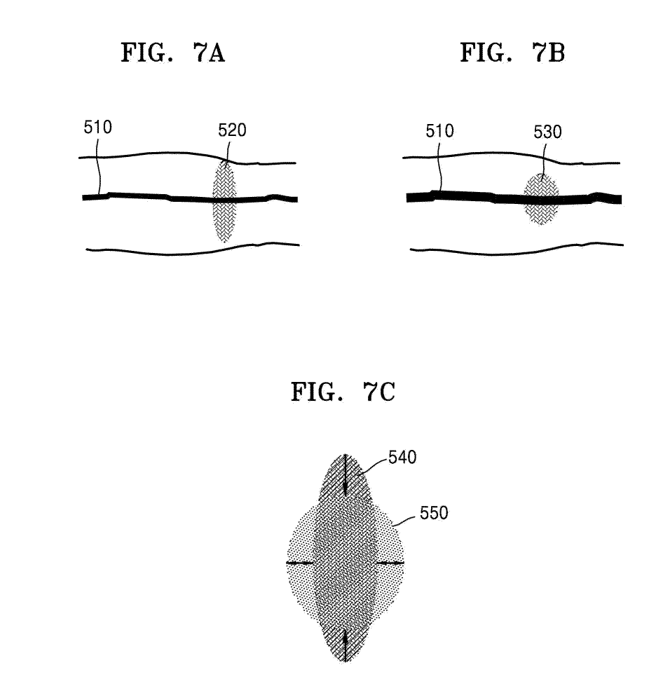

For example, the bio-information acquisition apparatus 100 may obtain a signal indicating a change in the volume of a blood vessel from the sensed laser speckle change, based on the correlation between the laser speckle change and the change in the volume of a blood vessel, which is described below in detail with reference to FIGS. 7A, 7B, and 7C.

FIGS. 7A, 7B, and 7C respectively illustrate a laser speckle distribution pattern (first pattern) 520 when a blood vessel 510 constricts, a laser speckle distribution pattern (second pattern) 530 when the blood vessel 510 dilates, and a comparison between the first pattern 510 and the second pattern 530, according to an exemplary embodiment. For example, referring to FIGS. 7A and 7B, when the blood vessel 510 constricts, the laser speckle distribution pattern 520 may indicate a lengthy oval, and when the blood vessel 510 dilates, the laser speckle distribution pattern 530 may indicate a circle.

The bio-information acquisition apparatus 100 may sense a change in the distribution pattern of a speckle according to the constriction and dilation of the blood vessel 510. For example, referring to FIG. 7C, in an area 540, a laser speckle may be distributed during the constriction of the blood vessel 510 and no laser speckle exists when the blood vessel 510 dilates. Accordingly, in the area 540, as the blood vessel 510 dilates, the intensity of the sensed laser speckle decrease and the laser speckle may be presented as a black dot.

In contrast, in an area 550, no laser speckle exists during the constriction of the blood vessel 510, and as the blood vessel 510 dilates, the laser speckle may be distributed. Accordingly, in the area 550, as the blood vessel 510 dilates, the intensity of the sensed laser speckle increases and the laser speckle may be presented as a white dot.

As such, the bio-information acquisition apparatus 100 may estimates a change in the distribution pattern of the laser speckle according to the change in the intensity of the sensed laser speckle. Also, the bio-information acquisition apparatus 100 may estimate a change in the volume of a blood vessel due to the constriction and dilation of a blood vessel, according to the change in the distribution pattern of a laser speckle.

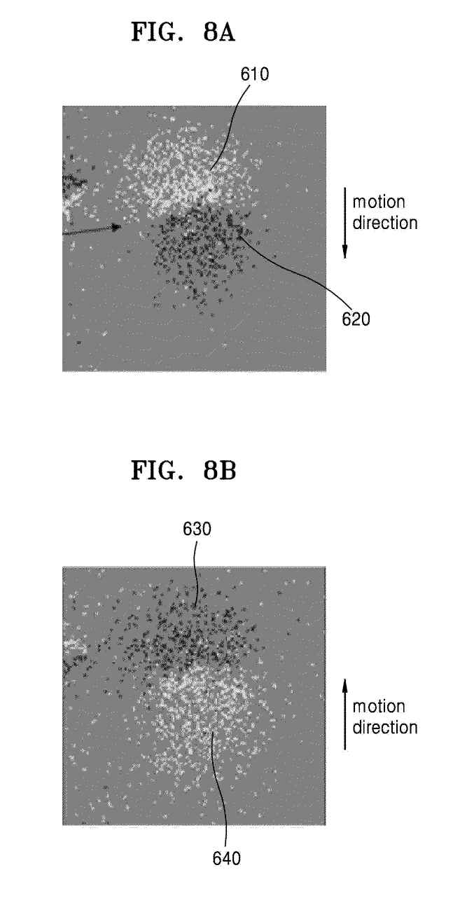

Also, the bio-information acquisition apparatus 100 may estimate a motion of a blood vessel according to the sensed laser speckle change, which is described below in detail with reference to FIGS. 8A and 8B.

FIGS. 8A and 8B illustrate changes in the laser speckle according to an exemplary embodiment. For example, as illustrated in FIG. 8A, when an object moves from up to down, the sensed laser speckle change is presented as a white dot in an upper area 610, and the sensed laser speckle change is presented as a black dot in a lower area 620.

In contrast, as illustrated in FIG. 8B, when an object moves from down to up, the sensed laser speckle change is presented as a white dot in a lower area 640, and the sensed laser speckle change is presented as a black dot in an upper area 630. The bio-information acquisition apparatus 100 may perform motion correction by using the above characteristics.

The bio-information acquisition apparatus 100 may obtain a waveform indicating the change in the volume of a blood vessel, by analyzing the change in the distribution pattern of a laser speckle.

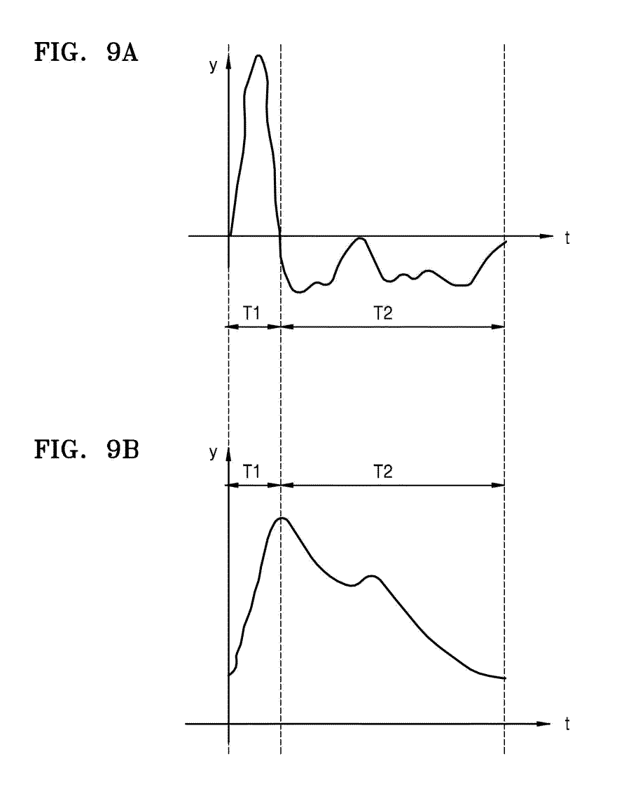

For example, FIGS. 9A and 9B are graphs respectively showing a waveform indicating a change in the volume of a blood vessel obtained in the bio-information acquisition apparatus 100 and a PPG waveform, that is, a waveform indicating the volume of a blood vessel. In the graph of FIG. 9A, the x axis denotes time t and the y axis denotes a change in the volume of a blood vessel. Also, in the graph of FIG. 9B, the x axis denotes time t and the y axis denotes the volume of a blood vessel. FIG. 9A may be a differentiated form of FIG. 9B and the waveform of FIG. 9B may be obtained by integrating the waveform of FIG. 9A.

The bio-information acquisition apparatus 100 may extract at least one parameter based on the obtained bio-signal waveform (operation S440), and may estimate a blood pressure by using the extracted parameters (operation S450).

The bio-information acquisition apparatus 100 may extract various parameters included in the obtained bio-signal waveform, and may estimate a systolic blood pressure and a diastolic blood pressure by applying the extracted parameters to a blood pressure estimation model. In this state, the blood pressure estimation model may be models formed based on a correlation between the extracted parameters and the systolic blood pressure or a correlation between the extracted parameters and a diastolic blood pressure. The blood pressure estimation model may include a linear model or a non-linear model. The non-linear model may include a neural network learning model or a model of comparing with a blood pressure measured by a cuff sphygmomanometer.

In an example, the bio-information acquisition apparatus 100 may estimate a blood pressure by applying parameters extracted from the waveform illustrated in FIG. 9A or 9B to a linear model. The linear model may be presented by Mathematical Formula 1 and Mathematical Formula 2 below.

SBP=a*T2+b [Mathematical Formula 1]

DBP=c*T2+d [Mathematical Formula 2]

In the above mathematical formulae, SBP denotes a systolic blood pressure or a maximum blood pressure, and DBP denotes a diastolic blood pressure or a minimum blood pressure. Also, “a” and “b” denote constants for calculating the systolic blood pressure, or constants that are determined according to a correlation between “T2” and the systolic blood pressure. Also, “c” and “d” denote constants for calculating the diastolic blood pressure, or constants that are determined according to a correlation between “T2” and the diastolic blood pressure. Also, “T2” denotes a diastolic time and may be extracted from the waveform illustrated in FIG. 9A or 9B. The bio-information acquisition apparatus 100 may estimate a systolic blood pressure and a diastolic blood pressure by applying a T2 value extracted from the waveform to Mathematical Formula 1 and Mathematical Formula 2.

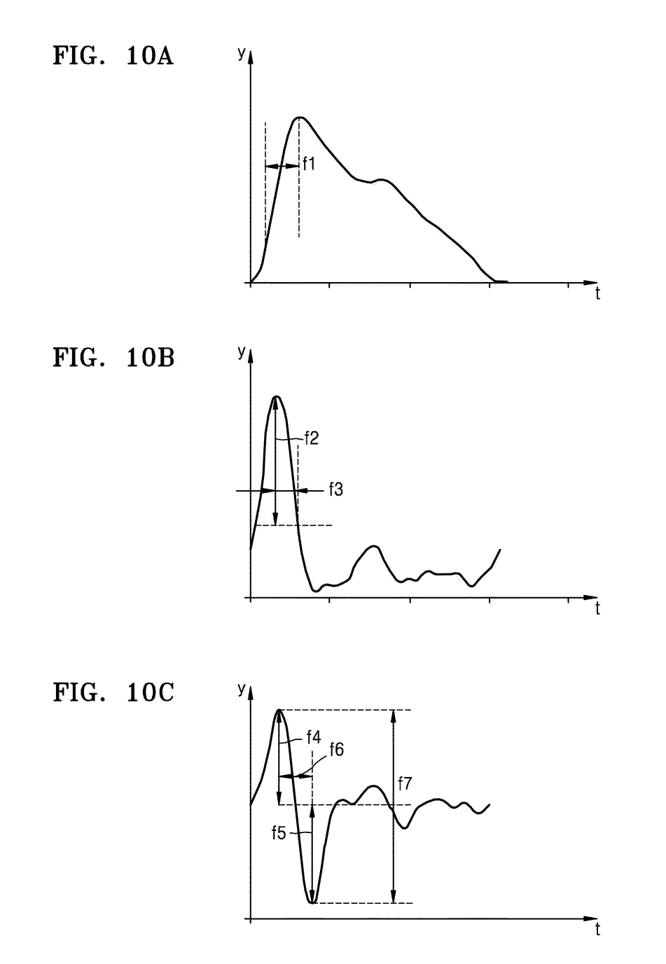

In another example, the bio-information acquisition apparatus 100 may estimate a blood pressure by applying parameters extracted from the waveform illustrated in FIGS. 10A to 10C to a linear model. FIG. 10A illustrates a waveform (first waveform) indicating the volume of a blood vessel, which is the same waveform of FIG. 9B. FIG. 10B illustrates a waveform (second waveform) indicating the change in the volume of a blood vessel, which is the same waveform of FIG. 9A. In other words, the waveform of FIG. 10B may be a waveform obtained by differentiating the waveform of FIG. 10A. Also, the waveform of FIG. 10C may be a waveform (third waveform) obtained by differentiating the waveform of FIG. 10B. In other words, the waveform of FIG. 10C may be a waveform (third waveform) obtained by differentiating the waveform of FIG. 10A twice. Although FIG. 10 illustrates only three waveforms for convenience of explanation, the present exemplary embodiment is not limited thereto and may further include a waveform obtained by differentiating again the waveform of FIG. 10C (waveform obtained by differentiating the waveform of FIG. 10A three times).

The bio-information acquisition apparatus 100 may extract at least one of parameters from each of the first to third waveforms. For example, f1 may be extracted from the first waveform, f2 and f3 may be extracted from the second waveform, and f4, f5, f6, and f7 may be extracted from the third waveform. Accordingly, the bio-information acquisition apparatus 100 may estimate a systolic blood pressure and a diastolic blood pressure by applying the extracted various parameters f1, f2, f3, f4, f5, f6, and f7 to a linear model.

Also, the bio-information acquisition apparatus 100 may analyze the change in the volume of a blood vessel in a frequency domain to be presented in a waveform according to a frequency, and may extract various parameters included in the waveform according to a frequency. Also, the bio-information acquisition apparatus 100 may estimate a systolic blood pressure and a diastolic blood pressure by applying the extracted parameters to a blood pressure estimation model in the waveform according to a frequency in the same method as the above-described waveform according to the time. In this state, the blood pressure estimation model may be models formed based on the correlation between the extracted parameters and the systolic blood pressure or the correlation between the extracted parameters and the diastolic blood pressure. For example, the blood pressure estimation model may be a linear model or a non-linear model.

In another example, the bio-information acquisition apparatus 100 may apply the parameters extracted from the bio-signal waveform to a neural network learning model. In detail, a neural network learning model regarding the estimation of a blood pressure is a model of outputting a final blood pressure matching the parameters input as queries by using a previously learned neural network data set when particular parameters are input as queries. In this state, the neural network data set may correspond to a sort of database that is previously learned through data mining regarding a correlation between the parameters in the bio-signal waveform and a blood pressure.

Accordingly, the bio-information acquisition apparatus 100 may input the parameters extracted from the waveforms illustrated in FIGS. 9A through 10C as queries of a neural network learning model, to obtain a final blood pressure from the previously learned neural network data set.

In addition, in order to estimate a blood pressure by using the bio-signal waveform, various well-known linear models or non-linear models may be used. Since methods of using various linear models or non-linear models are obvious to one of ordinary skill in the art, detailed descriptions thereof are omitted.

FIG. 11 is a flowchart for explaining a method of acquiring information about a blood flow according to an exemplary embodiment.

Since operation S710 and operation S720 of FIG. 11 are substantially the same as operation S310 and operation S320 of FIG. 4, detailed descriptions thereof are omitted.

The bio-information acquisition apparatus 100 may extract information about a blood flow, by using a laser speckle change (operation S730).

While a CMOS or CCD type image sensor senses a motion of a blood flow, the bio-information acquisition apparatus 100 including the DVS senses a laser speckle change and thus a change in a velocity or direction of a blood flow, that is, an acceleration of a blood flow may be sensed. For example, since the DVS senses only a case when the strength of an optical signal corresponding to the laser speckle changes, the acceleration of a blood flow, for example, the change in the velocity or direction of a blood flow, may be sensed.

For example, when the velocity of a blood flow changes, a distribution of a laser speckle changes in an area where the velocity changes, and thus the strength of an optical signal corresponding to the laser speckle may change. Accordingly, the bio-information acquisition apparatus 100 may obtain a change in the distribution of a laser speckle by sensing a change in the intensity of an optical signal corresponding to the laser speckle by using the DVS.

The bio-information acquisition apparatus 100 may sense a change in the spatio-temporal distribution pattern of a laser speckle, based on a change in the intensity of an optical signal corresponding to the sensed laser speckle. In this state, the spatio-temporal distribution pattern of a laser speckle may denote a pattern in which the laser speckle sensed from the ROI is distributed spatially and temporally. For example, when no motion such as a blood flow exists in the ROI, the distribution pattern of a laser speckle may not change spatially and temporally and the laser speckle may be distributed in the same pattern.

In contrast, when a motion exists in the ROI, the distribution pattern of a laser speckle changes spatially and temporally, the bio-information acquisition apparatus 100 may sense a change in the distribution pattern of a laser speckle.

The bio-information acquisition apparatus 100 may analyze the spatio-temporal correlation with respect to the ROI and may estimate the accelerative change of a blood flow, by using a change in the distribution pattern of a laser speckle. A variety of algorithms are used for analyzing the spatio-temporal correlation. The bio-information acquisition apparatus 100 may obtain the spatio-temporal correlation by inputting a signal corresponding to the sensed laser speckle change to a spatio-temporal correlator.

The bio-information acquisition apparatus 100 may estimate an accelerative change of a blood flow by using the spatio-temporal correlation in the ROI. For example, as a degree of the spatio-temporal correlation decreases, the accelerative change may be estimated to be larger. As a degree of the spatio-temporal correlation increases, the accelerative change may be estimated to be smaller.

Also, the bio-information acquisition apparatus 100 may preset a distribution of the estimated accelerative change in a two-dimensional image, and may obtain a distribution of the velocity of a blood flow based on the accelerative change of a blood flow.

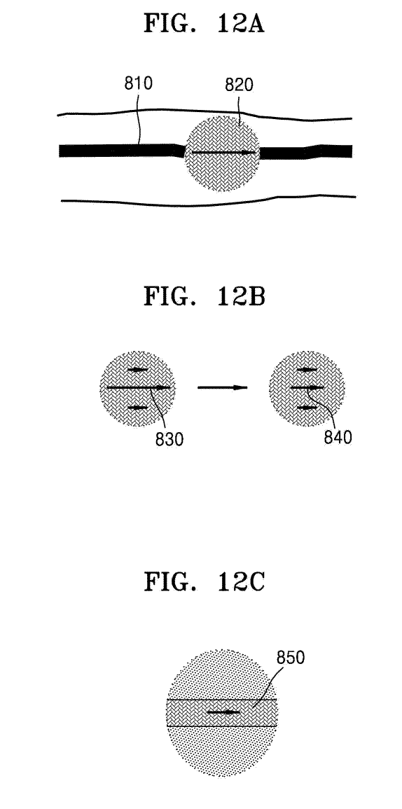

FIGS. 12A, 12B, and 12C are views for explaining a method of acquiring information about a blood flow according to an exemplary embodiment.

As illustrated in FIG. 12A, the bio-information acquisition apparatus 100 may measure a change of a distribution pattern of a laser speckle 820 by radiating a laser beam to the ROI including a blood vessel.

For example, as illustrated in FIG. 12B, when a first flow 830 of a blood flow changes to a second flow 840 in a first area, that is, an acceleration of a blood flow exists, for example, the velocity or direction of the blood flow changes, the distribution pattern of a laser speckle in the first area changes. As shown in FIG. 12C, when the first flow 830 is changed to the second flow 840, the strength of an optical signal may change in the first area that is an area where a change in the blood flow exists, and since no flow exists or no change in the flow exists in the other area, the strength of the optical signal does not change.

Accordingly, the bio-information acquisition apparatus 100 may sense a laser speckle change corresponding to the change in the intensity of an optical signal in a first area 850, by using the DVS, as illustrated in FIG. 12C, and may sense a change in the distribution pattern of a laser speckle in the first area 850.

The bio-information acquisition apparatus 100 may analyze the spatio-temporal correlation with respect to the ROI and may estimate an acceleration of a blood flow, by using a change in the sensed distribution pattern of a laser speckle.

The bio-information acquisition apparatus 100 may estimate the acceleration of a blood flow by using the spatio-temporal correlation in the ROI. For example, as a degree of the spatio-temporal correlation decreases, the acceleration may be estimated to be larger. As a degree of the spatio-temporal correlation increases, the acceleration may be estimated to be smaller. However, the present exemplary embodiment is not limited thereto.

As described above, according to the one or more of the above embodiments, bio-information of a user may be measured in real time in a cuff-less method based on a motion of a living body, and remotely, user convenience may be improved.

Since an image processing is performed only when a motion is sensed, an amount of power consumption may be reduced and a long-time measurement may be facilitated.

Also, information of a two-dimensional image may be processed with a minimum amount of digital information. Accordingly, bio-information of a user may be measured in real time and remotely.

The computer readable code may be recorded and/or transferred on a medium in a variety of ways, and examples of the medium includes recording media, such as magnetic storage media (e.g., read only memory (ROM), floppy disks, hard disks, etc.) and optical recording media (e.g., compact disc read only memories (CD-ROMs), or digital versatile discs (DVDs)), and transmission media such as Internet transmission media. Thus, the medium may have a structure suitable for storing or carrying a signal or information, such as a device carrying a bitstream according to one or more exemplary embodiments. The medium may also be on a distributed network, so that the computer readable code is stored and/or transferred on the medium and executed in a distributed fashion.

The foregoing exemplary embodiments are merely exemplary and are not to be construed as limiting. The present disclosure can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for acquiring bio-information, the apparatus comprising:
a light source configured to radiate a laser beam to a region of interest including a blood vessel;
a sensor configured to sense, from the region of interest, a change of a laser speckle generated by the radiated laser beam; and
a controller configured to obtain a bio-signal indicating a change in a blood flow in the blood vessel based on the sensed change of the laser speckle.

2. The apparatus of claim 1, wherein the sensor is further configured to sense whether a strength of an optical signal corresponding to the laser speckle increases or decreases.

3. The apparatus of claim 1, wherein the sensor comprises a dynamic vision sensor (DVS).

4. The apparatus of claim 1, wherein the sensor is further configured to enter a standby mode in response to the change of the laser speckle not being sensed for a predetermined time.

5. The apparatus of claim 1, wherein the controller is further configured to estimate a blood pressure based on the obtained bio-signal.

6. The apparatus of claim 5, wherein the controller is further configured to generate a waveform indicating a change in the volume of the blood vessel based on the change in the blood flow and the sensed change of the laser speckle and determine a blood pressure of the blood vessel based on at least one of parameters of the waveform.

7. The apparatus of claim 6, wherein the controller is further configured to determine the blood pressure by applying the at least one of parameters to a blood pressure estimation model, and the blood pressure estimation model is generated based on a correlation between the at least one of parameters and a reference blood pressure.

8. The apparatus of claim 5, wherein the controller is further configured to generate a waveform indicating a change in a volume of the blood vessel based on the sensed change of the laser speckle, generate a photoplethysmogram (PPG) waveform indicating the volume of the blood vessel based on the generated waveform, and determine the blood pressure based on the PPG waveform.

9. The apparatus of claim 1, wherein the controller is further configured to determine an acceleration of the blood flow based on a correlation between the sensed change of the laser speckle and a reference blood flow acceleration.

10. The apparatus of claim 9, further comprising a display configured to display an image indicating the determined acceleration of the blood flow.

11. A method of acquiring bio-information, the method comprising:
radiating a laser beam to a region of interest (ROI) including a blood vessel;
sensing a change of a laser speckle generated by the radiated laser beam, from the region of interest; and
obtaining a bio-signal indicating a change in a blood flow in the blood vessel based on the sensed change of the laser speckle.

12. The method of claim 11, wherein the sensing the change of the laser speckle change comprises sensing whether a strength of the laser speckle is increased or decreased.

13. The method of claim 11, further comprising entering a standby mode in response to the change of the laser speckle not being detected for a predetermined period of time.

14. The method of claim 11, further comprising determining a blood pressure of the blood vessel based on the obtained bio-signal.

15. The method of claim 11, further comprising:
generating a waveform indicating a change in a volume of the blood vessel based on the change in the blood flow and the sensed change of the laser speckle; and
determining a blood pressure of the blood vessel based on at least one of parameters of the waveform.

16. The method of claim 15, the determining the blood pressure comprises applying the at least one of parameters to a blood pressure estimation model, wherein the blood pressure estimation model is generated based on a correlation between the at least one of parameters and the blood pressure.

17. The method of claim 11, further comprising:
generating a waveform indicating a change in a volume of the blood vessel based on the sensed change of the laser speckle;
generating a photoplethysmogram (PPG) waveform indicating the volume of the blood vessel based on the waveform; and
determining a blood pressure based on the PPG waveform.

18. The method of claim 11, further comprising determining an acceleration of the blood flow based on a correlation between the sensed change of the laser speckle and a reference blood flow acceleration.

19. The method of claim 18, further comprising displaying an image indicating the estimated acceleration of the blood flow.

20. A non-transitory computer readable storage medium storing a program that is executable by a computer to perform the method of claim 11.

21. An apparatus for obtaining bio-information, the apparatus comprising:
an optical sensor configured to capture an image of a skin surface of a subject within a region of interest (ROI) and generate data on a portion of the image that has a relative change of a light intensity in relation to a remaining portion of the image; and
a controller configured to obtain the bio-information indicating a change in a blood vessel within the ROI based on the generated data.

22. The apparatus of claim 21, wherein the optical sensor comprises a dynamic vision sensor (DVS) and the generated data reflects the change of the light intensity which is caused by a blood flow of the blood vessel within the ROI.
